(19) United States
(12) Patent Application Publication
(10) Pub. No.: US 2019/0328227 A1
(43) Pub. Date: Oct. 31, 2019

(54) INTERACTIVE SCHEDULER AND MONITOR

(71) Applicant: ICAN INTERACTIVE INC., Maple (CA)

(72) Inventors: STEPHEN EADES, Maple (CA); ROSS BIGELOW, Barrie (CA); TRISTAN WILSON, Wairton (CA); WESLEY FLYNN, Stayner (CA)

(21) Appl. No.: 16/398,392

(22) Filed: Apr. 30, 2019

Related U.S. Application Data
(60) Provisional application No. 62/664,433, filed on Apr. 30, 2018.

(51) Int. Cl. A61B 5/00; G16H 10/60

(52) U.S. Cl. CPC A61B 5/002; A61B 5/0024; G16H 10/60; A61B 5/743; A61B 5/72

(57) ABSTRACT

A personal management device includes a programmable scheduler to permit a user's schedule for a designated period to be recorded. A clock function provides prompts for each scheduled activity at an appropriate time. The device also includes a monitor of biological indicators and upon attainment of threshold levels indicative of onset of anxiety, interrupts the scheduled activity to implement a coping strategy.

### FIELD OF THE INVENTION

The present invention relates to an interactive scheduler and monitor.

### DESCRIPTION OF THE PRIOR ART

The organisation of tasks within a particular time frame is a facet of everyday life. However, for certain people this is a challenge that can give rise to anxiety and potentially lead to onset of adverse situations such as behavioural issues. Although this challenge applies to people in all age groups, a particular group of people who are prone to these challenges are children and youth with Autism Spectrum Disorder (ASD), many of whom often experience anxiety and struggle with self-regulation. It has been proposed that struggles with emotional regulation might explain both the degree of externalising behaviours, such as anger and aggression, and internalising behaviours, such as anxiety and depression, among youth with ASD.

It has been recognised that those affected with ASD require additional support to fully integrate in to the community and reach their full potentials. Professionals across health care, education and social services have designed many tools, interventions and strategies to assist people with ASD and individualised programming with real time feedback, encouragement and guidance has been found to lead to more positive outcomes.

The delivery of such programs is however labour intensive and over the course of a typical day may require the involvement of several professionals, each of who must be familiar with the individuals under their supervision and be prepared to respond in an appropriate manner. This of course is onerous and may inhibit utilisation of the tools available.

Similar considerations apply in the treatment of other conditions such as ABI (Aquired Brain Injury), Dementia syndrome (i.e. Alzheimer's disease, and other memory related diseases or disorders), where a personalised intervention can lead to positive results but requires significant investment in human resources.

It is therefore an object of the present invention to obviate or mitigate the above disadvantages.

### SUMMARY OF THE INVENTION

In general terms, the present invention includes A personal management device that includes a programmable scheduler to permit a sequence of events corresponding to a users coping strategy to be recorded. A discernible user interface is provided and a monitor to monitor biometric indicators. The monitor responds to attainment of threshold levels of the biometric indicators indicative of onset of an adverse situation to recall the sequence of events and implement the coping strategy through said user discernible interface. Preferably, implementation of the coping strategy is accompanied by an alert to the supervising person.

Preferably also the programmable scheduler enables a schedule of activities for a user for a designated period to be recorded and the monitor interrupts the schedule for the designated period upon attainment of said threshold levels.

As a further preference, completion of a scheduled activity provides a reward to the user.

In a preferred embodiment, completion of the coping strategy returns the scheduler to its scheduled activity.

The scheduling device is preferably a wearable device having a discernible user interface such as a viewable screen or a voice assistant to prompt the required activity. The biometric indicators may include pulse, breathing rate, blood pressure, body temperature, skin condition such as perspiration and rate of movement that are integrated in to the wearable device. The device includes a geo-fencing function to provide indications of location and communicates wirelessly with supervisory personnel.

### BRIEF DESCRIPTION OF THE DRAWINGS

An embodiment of the invention will now be described by way of example only with reference to the accompanying drawings wherein:

FIG. 1 is a schematic representation of a user network incorporating a personal management device

FIG. 2 is a functional diagram of the architecture of the personal management device;

FIG. 3 is a flow chart showing the operation of the personal management device under normal circumstances

FIG. 4 is a flow chart similar to FIG. 3 showing intervention of a coping strategy,

FIG. 5 is a screen shot of different granularities of instruction available;

FIG. 6 is a flow chart similar to FIG. 3 of a further embodiment of personal management device; and

FIG. 7 is a flow chart of a yet further embodiment of personal management device.

### DETAILED DESCRIPTION OF THE INVENTION

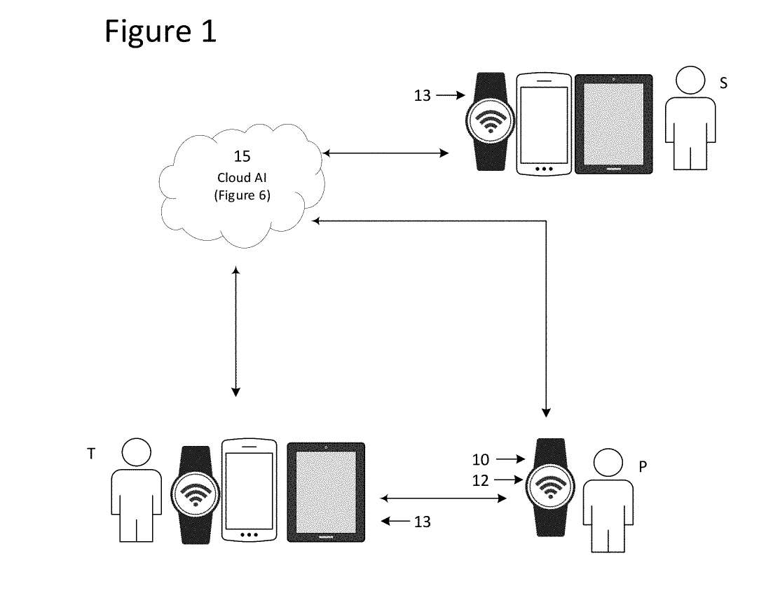

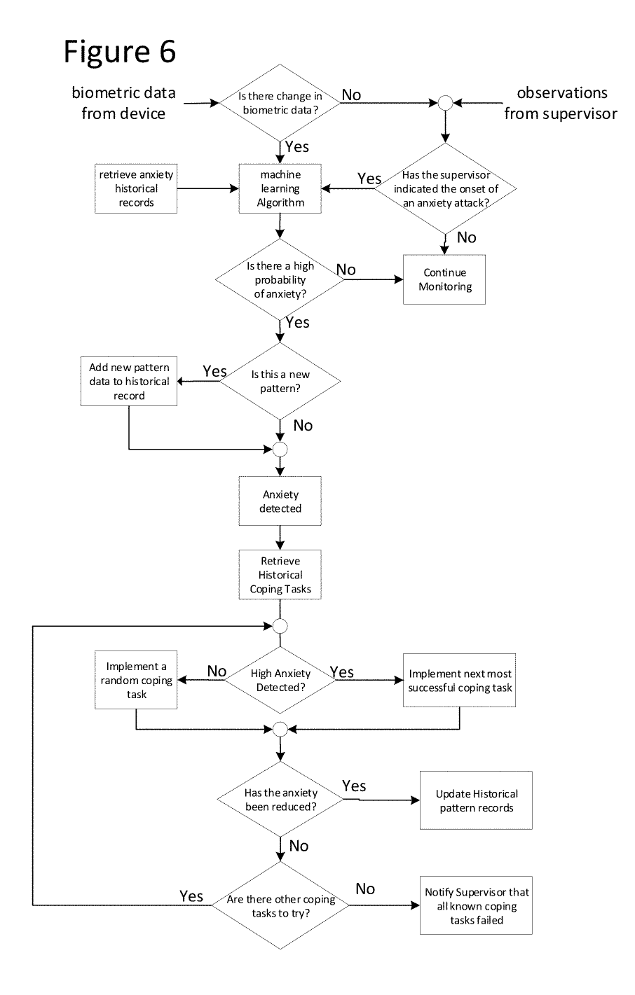

Referring therefore to FIG. 1, a user P has a wearable device 10 that is conveniently in the form of a watch to be worn on the wrist or similar device. The device 10 has a communication module 12 that can communicate via wireless communication protocols such as Bluetooth, wi-fi, cellular or near field communication protocols (NFC) with an adjacent supervisor T and for communication requiring greater range through cellular or wi-fi communication with more distant supervisors S. Each of the supervisors T, S have a computing device 13, such as a computer, smart phone, tablet or wearable that implements a program or app to monitor the device 10. Communication may be conducted through the “Cloud” 15 to offload certain functions from the computing devices 10, 13, as will be explained more fully below particularly with respect to FIG. 6.

In a typical environment, the user P may be a child exhibiting behavioural traits associated with ASD and the supervisor T is a teacher supervising a class that includes the child P. The distant supervisor S is a parent who has overall responsibility for the child P. It will be appreciated that the relationships and responsibilities of the supervisors T, S to the user P are dynamic and will vary over a typical day, for example before, during and after school and between weekdays and weekends.

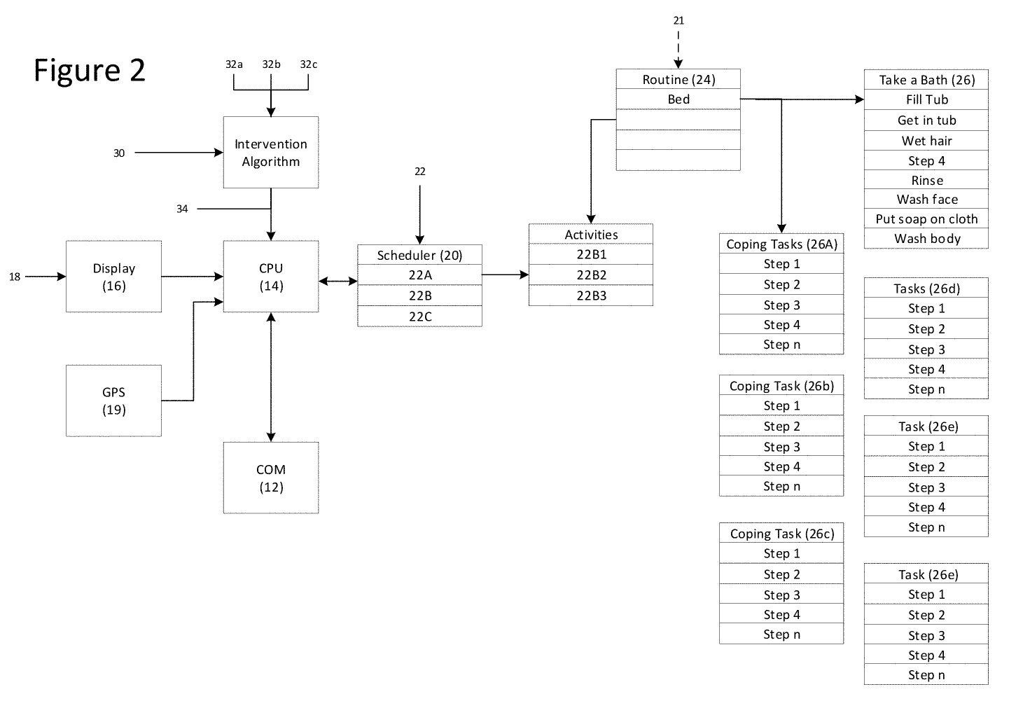

The device 10 is shown schematically in FIG. 2, and has a CPU 14 that drives a discernible user interface, preferably a viewable display 16, and controls the communication module 12. The display 16 has a user input 18, which is typically a touch screen function, to provide information to the CPU 14, and a GPS function 19 that provides location information to the CPU 14. In certain situations, a viewable display may not be not practical, such as where the user has impaired vision or cannot comprehend written directions. In this case an audible interface may be utilised as the user discernible interface. Such interfaces provide a virtual voice assistant allowing querying by the user P and the issuance of audible responses and directions. Examples of such interfaces are known by there trade names “Siri”, “Alexa” and “Google Home”, depending on the platform utilised.

The CPU 14 communicates with a scheduler 20 that is implemented in RAM 21 and is programmable to allocate activities to specific time intervals 22. The scheduler 20 has a clock function and may coarsely divide each day to “before school” 22A, “school” 22B and “after school” activities 22C. The basic allocations may be more finely divided in to specific activities at designated time intervals so that for example the school allocation 22B can be sub-divided in to individual lesson periods, 22B1; 22B2; 22B3 etc.

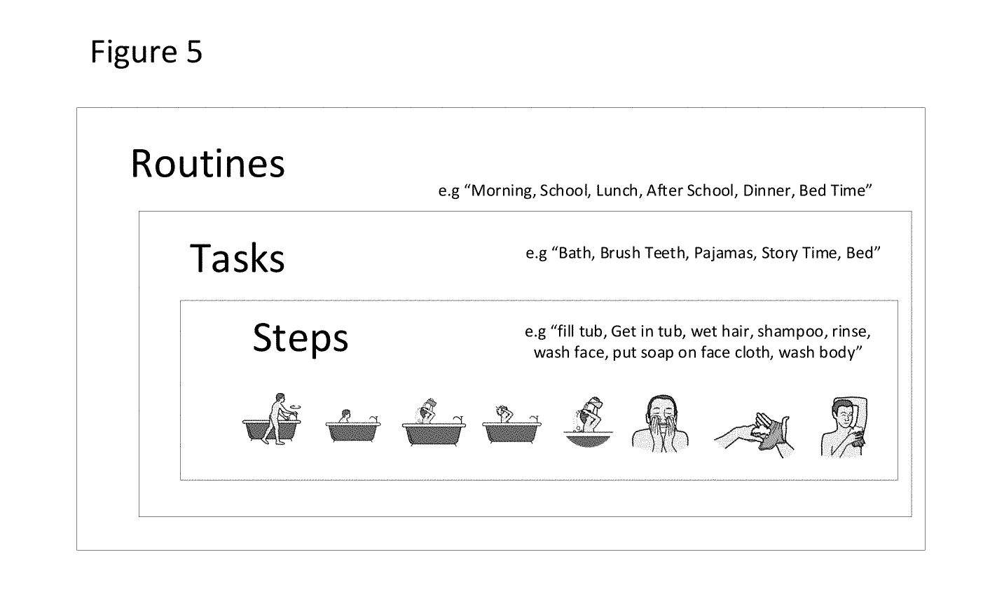

Each of the activities is compiled from one or more routines 24 stored in the memory 21, and in turn the routines may themselves be subdivided in to specific sub-routines, referred to as tasks 26. As illustrated in in FIG. 5, the post school activity 22C includes a routine of “bed time” which is imported from the routines 24 in to the scheduler 20 at the designated time within the time interval 22C. The bed time routine sets out the tasks of “bath, brush teeth, pajamas, story time, bed” and each task is linked to individual steps that may be displayed pictorially on the viewable display 16. Each task and step includes graphic files that are loaded sequentially on the display 16 to provide direction to the user P. Thus, as shown in FIG. 5, when the routine of “bed time” is invoked by the scheduler, the task “Bath” is recalled and the steps designated for “Bath”, namely fill tub, get in tub, wet hair etc. are displayed on the viewable display 16. Upon completion of that task, subsequent tasks, “brush teeth” etc. are called in sequence as indicated by tasks 26d, 26e, 26f. Each of those tasks will have steps associated with them and instructions to be provided through the discernible interface.

Where appropriate, each routine or task will have a specific time period associated with it, e.g. “bed” of 30 minutes subdivided by tasks 26 of a bath of 10 minutes, brush teeth 2 minutes, putting on pajamas of 2 minutes, etc. Each task may similarly allocate set times for each step within the task, for example, filling tub 3 minutes. Upon lapsing of the allocated time for each step, a discernible indicator, such as a “beep”, is provided to advise the user P to advance to the next step. Upon completion of all steps, the user P indicates completion by the input 18 and the steps of the next task are displayed.

Where the activity is not time based, the user may indicate completion of the task by the input 18, and reminders may be sent to advise the user P to indicate when the task is completed. In some circumstances, completion of a task may be indicated by attainment of a designated location as determined by a GPS function 19.

The scheduler 22 and the routines and tasks are programmable by the supervisor S, T who has the administrative authority for that period so that individual schedules may be developed to suit the user P. Common routines and tasks may be preloaded and stored in memory 21 and selected as needed on the smart phone 13 of the supervisor S, T. Custom routines can be prepared on the smart phone 13 of the supervisor S using a graphic programming tool for particular circumstances. The custom routines may be modifications to existing preloaded routines or entirely original programmed by the supervisor S. The routines and tasks are downloaded from the supervisor's smart phone 13 to the users device 10 for storage in the memory 21. A schedule is thus compiled for a particular time period from one or more of the supervisors S, T who have administrative authority at different times of the day. The supervisors are organised hierarchically with the supervisor S setting the overall structure for the day but ceding authority to, for example, the teacher T for periods that have been designated as school periods.

The routines 24 also include an intervention routine that invokes one or more a coping tasks 26A, 26B, 26C, each of which implement a sequence events to implement a coping strategy. A coping strategy is a sequence events organised as tasks and associated steps found to be effective in reducing behavioural issues resulting from external factors, such as over stimulation, or internal factors, such as anxiety. Each coping strategy, such as the coping task 26A, will be personal to the user P based on experience and is programmed by the supervisor S as a custom routine. Such a coping strategy may include, for example, a direction to move to a quiet zone, playing a particular musical piece or walking along a defined route. The coping strategy is implemented as a task having steps with graphic files for display on the display 16.

Multiple coping strategies may be developed to address different situations and each strategy may be defined through a different task, 26A, 26B, 26C etc. and implemented as determined by the prevailing condition.

To implement an appropriate one of the coping tasks 26A, 26B, 26C, the CPU 14 is also connected to an intervention algorithm 30 that receives inputs from one or more biometric sensors 32. The biometric sensors 32 monitor parameters that collectively are indicative of onset of stress. Those parameters include heart rate or pulse, 32a, skin condition (perspiration) 32b and activity level 32c determined by rate of movement measured by an accelerometer. Other biometric parameters might include breathing rate, blood pressure, body temperature. The biometric sensors 32 are integrated in to the wearable device 10 and the intervention algorithm 30 combines the readings as will be discussed more fully below to determine whether the parameters approach a threshold that indicates that onset of a behavioural issue is imminent. If a threshold is exceeded, an interrupt signal 34 is provided to the CPU 14, which interrupts the routine that is being illustrated by steps on the display 16 and replaces it with the tasks and steps associated with one of the coping strategies 26A-C. The intervention algorithm 30 continues to monitor the user's P physiological condition and when it returns to normal and the selected one of the coping tasks 26A-C is completed, the interrupt signal 34 is removed and the scheduler 10 reverts to its previous condition.

The interrupt algorithm 30 will be determined by the characteristics of the user P and as such will vary from user to user. Further, as noted above, there may be multiple algorithms that apply to a particular user P and any one of those may trigger the interrupt signal 34 and call a particular one of the coping tasks 26A-C. By way of example, an elevated heart rate in excess of 90 beats per minute for an extended period may indicate onset of a critical condition and therefore implementation of coping task 26A is appropriate. Thus, the intervention algorithm 30 triggers an event if the heart rate is >90 for 2 minutes and indicates that task 24a should be implemented.

In an alternative situation, an increased activity level and an elevated heart rate may be indicative of onset of a condition requiring intervention. In this case rapid repetitive movement of the user's hand is monitored by the biometric sensor 32c and combined with the heart rate to determine the threshold. Therefore, if the heart rate exceeds 80 AND the activity level exceeds 70% THEN an interrupt signal 34 is provided to select the coping task 26B. The interrupt algorithm 30 continues to monitor the sensors 32 and if an elevated condition persists another intervention strategy, 26C, is triggered. If the sensors determine that the coping task has lowered the physiological conditions to acceptable levels, the coping task is replaced by the previous task.

The intervention algorithm 30 may also incorporate an input from the selected routine 24 in the scheduler 20 to modify the threshold. For example, if bath time is the current routine, the input from the skin condition sensor 32b may be modified to take in to account the user P is in the bath, or if the scheduled event is a gym class the heart rate monitor 32a may be modified accordingly.

Device 10 also includes a GPS utility 19 to provide a geo-positioning signal of the user P. The geo-positioning signal may be used by the intervention algorithm to provide an appropriate coping strategy. If the biometric sensors determine an elevation of stress levels, the geo-positioning signal is examined and compared with the anticipated position of the user P for the scheduled routine. For example, if the scheduler indicates a return from school, and the routine indicates a geofence for that activity, an examination of the actual position may show that the user P has departed from the expected path and effectively is lost. The CPU 14 provides direction on the display 16 to return the user to the correct location and remove the stress condition from the user, or instruction to find help or for example to go in to a shop for assistance. The task may include a step of displaying on the display 16 a message, such as “I am lost, please tell me how to get to 123 ABC St.” or a phone number to call to assist in repatriation.

In each of these scenarios, it will be appreciated that the device 10 monitors the user's P biometric indicators and determines that the onset of an adverse situation such as a behavioural issue is imminent. In response to that determination, and intervention strategy is invoked and provided to the user P to assist in alleviation of the condition.

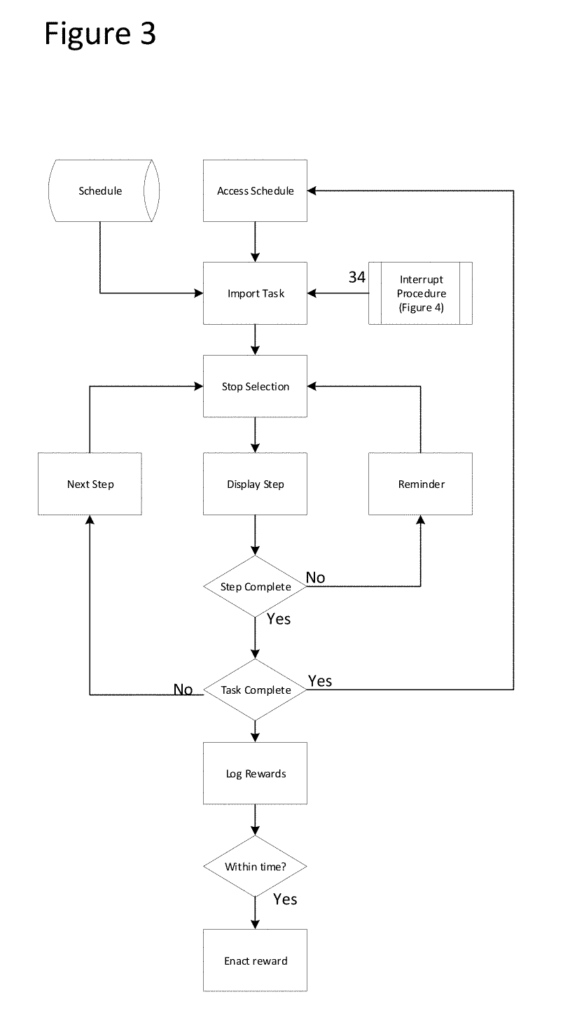

In use, as indicated in FIG. 3, the user P accesses the scheduler 20 to determine the first activity. The first routine 24 is imported and the first task displayed on the display 16. After the predetermined time, the display 16 looks for an indication the task is complete. This may be from the input 18 or from a sensed condition, such as the movement of the user P to a different location.

If the Task is complete, the CPU determines if another task 26 is required in the routine 24. If there is no further task 26, the routine 24 is considered complete and the scheduler 20 notified. The next routine 24 is then loaded. At the same time, the completion of the task 26 is recorded in a log and a reward point allocated to the user. The reward point is stored within the memory 21 and points are accumulated and may be redeemed through the supervisor S for discretionary benefits. A positive reinforcement of the benefits of completing the tasks 26 is thus provided.

If the task 26 is not completed in the set time, a reminder message, or fallback message, is generated on the display 16 to encourage completion of the task.

If another task 26 is required in the routine 24, an incrementing message is sent to select the next task 26 in the routine 24 and display the appropriate message on the display 16.

Where a routine 24 is completed ahead of the allotted time, a reward routine is called, such as allowing access to a video game or video for the free time until the next routine is scheduled and thereby positively reinforce productive activities. The scheduler 20 will thus proceed through the schedule to assist in setting out tasks 26 that will complete routines 24 and meet the activities scheduled.

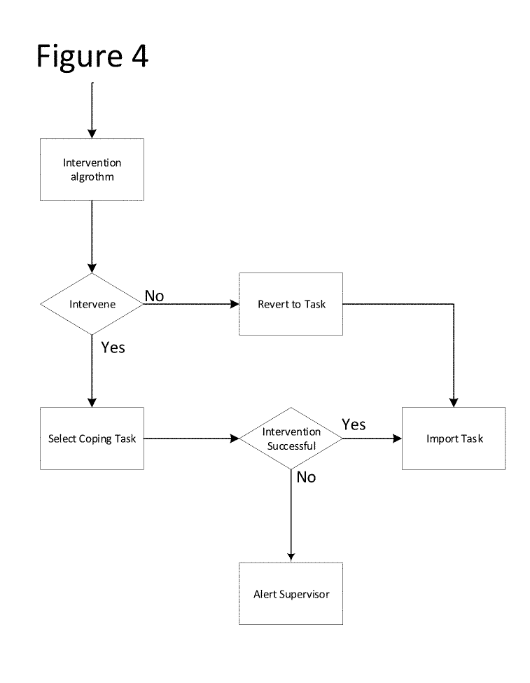

If, however, during the activities the onset of a stressful condition is detected, the interrupt signal 34 is provided to the CPU 14 and the coping routine 26A-C implemented as shown in FIG. 4. At the same time, the supervisor T is alerted through the communication module 12 as to the potential onset of the critical condition and may be attentive to it. Assuming the coping task 26A-C invoked is effective, the interrupt signal 34 is removed and the scheduler 20 reverts to the previous routine. In some cases, there may be insufficient time to revert to the routine 24 and so an alternative routine is selected, such as reading a book, until the next routine is implemented.

If the coping strategy embodied in the coping task 26A-C selected is not effective, the supervisor T may directly intervene, and the other supervisor S can be notified through the communication module 12 for a further resource.

It will be seen therefore that the scheduler 10 may be used to assist in accomplishing everyday schedules and the implementation of the coping task may mitigate the occurrence of stressful conditions that lead to more severe behavioural issues.

Although the specific example discussed above is directed to ASD, it will be appreciated that some or all of the features described may be applicable to other neurological conditions such as dementia etc. The coping strategy might include the display of certain images and the playing of a particular piece of music or direction to a room or location that provides security to the user P. These would be incorporated in to coping tasks 26A-C and invoked upon detection of the onset of a behavioural issue. In such an environment, where anxiety may be caused by failure to remember how to do things or where items are kept, the initial step of the coping task 26A may provide options for the user to select the task at hand and the strategy invoked will provide guidance.

A further embodiment of the device 10 is shown in FIG. 6. In this embodiment, machine learning algorithms are utilised to improve continuously the detection of the onset of behavioural issues and to improve the coping strategies implemented. The machine learning algorithms utilised will depend on the nature of the data received and utilised and commonly algorithms such as Linear and Logistical regression, Linear discriminant analysis regression trees, Naïve Bayes, K-Nearest Neighbour, Learning Vector Quantization and Random forest may be utilised.

The computing power available in the cloud 15 is used to modify and update detection and coping strategies that are communicated to the user P and the supervisors S, T.

Referring to FIG. 6, the biometric data from the monitors 32 is transmitted to the computing assets in the cloud 15. Messages from the supervisor T are also provide to the cloud 15. The incoming data from the sensors 32 is compared with previous data to determine whether there is a significant change. If not, and if there is no indication of anxiety from the supervisor, no action is taken.

If however a change is detected, or if the supervisor has indicated the onset of an anxiety attack or other behavioural issue, a machine learning algorithm (MLA) is invoked. The MLA retrieves the historical records of the user P relating to previous anxiety attacks and determines whether there is a high probability of anxiety from a comparison of the current data to historical data. If there is not a high probability, the monitoring continues.

If the MLA determines there is a high probability, it then determines whether this is a new pattern. If so, the new pattern is added to the historical record and a determination made that onset of an anxiety attack has been detected. Records of historical coping tasks 26A are then retrieved.

A determination is then made as to the degree of anxiety detected. If it is not considered high, a random coping task is implemented and the biometric parameters monitored for improvement. If there is improvement, the historical records are updated to associate that coping strategy with the detected pattern.

If no improvement is detected, a different coping task is selected and the results monitored for improvement. Where no other coping task is available, the supervisor T is notified so direct intervention can be implemented.

If a high degree of anxiety is detected, the most successful coping task from the historical records is selected. The effect of this is monitored and if successful is added to the records. If it is not successful, other strategies are selected, or the supervisor notified.

By continuously updating the historical records to match patterns with coping tasks, the efficacy of the coping tasks is enhanced.

The implementation of the MLA within the cloud 15, permits the experiences of other users to be be utilised to propose and select a new coping task from experiences of other users and adapt that coping task to the particular user. However, the data contained in the individual records is sensitive and appropriate steps have to be taken to ensure confidentiality.

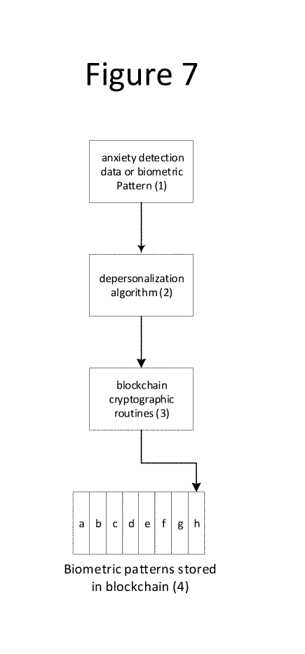

FIG. 7 shows schematically one such implementation intended to permit sharing of experiences whilst maintaining the required degree of confidentiality.

Referring to FIG. 7, when an anxiety incident or other interesting biometric shift is detected (101) it is sent to a block chain system (104) to improve the capabilities and increase the accuracy of detecting future anxiety incidents by sharing data collected from numerous biometric sensors. The data is processed by a depersonalization algorithms (102) and established blockchain cryptographic routines (103) and provide a secure, distributed, irrevocable and tamper resistant data store that can be shared with researchers to who can further analyze patterns and trends for various neurological conditions.

Although the invention has been described with reference to certain specific embodiments, various modifications thereof will be apparent to those skilled in the art without departing from the spirit and scope of the invention as outlined in the claims appended hereto. The entire disclosures of all references recited above are incorporated herein by reference.

What is claimed is:

1. A personal management device that includes a programmable scheduler to permit a sequence of events corresponding to a users coping strategy to be recorded a discernible user interface and a monitor to monitor biometric indicators, said monitor responding to attainment of threshold levels of said biometric indicators indicative of onset of an adverse situation to recall said sequence of events and implement said coping strategy through said user discernible interface.

2. The device of claim 1 wherein implementation of the coping strategy is accompanied by an alert to the supervising person.

3. The device of claim 1 wherein said scheduler enables a schedule of activities for a user for a designated period to be recorded, said monitor interrupting said schedule for said designated period upon attainment of said threshold levels.

4. The device of claim 3 wherein completion of a scheduled activity provides a reward to the user.

5. The device of claim 3 wherein completion of the coping strategy returns the scheduler to its scheduled activity.

6. The device of claim 1 wherein the programmable scheduler is a wearable device having a discernible user interface to prompt the required activity.

7. The device of claim 6 wherein said discernible interface is a viewable screen.

8. The device of claim 6 wherein the biometric indicators include at least one of pulse, breathing rate, blood pressure, body temperature, skin condition such as perspiration and rate of movement that are integrated in to the wearable device.

9. The device of claim 1 including a geo-fencing function to provide indications of location and to provide directions to a desired location through said discernible user interface.

10. The device of claim 3 wherein said sequence of activities is organised as routines with each routine comprised of one or more tasks with each task itself comprising one or more steps.

11. The device of claim 10 wherein said steps are communicated to said user through said discernible user interface.

12. The device of claim 11 wherein said discernible user interface is a visible display.

13. The device of claim 12 wherein said steps are communicated as pictograms.

14. The device of claim 11 wherein said coping strategy is implemented as a routine having designated tasks and steps.

15. The device of claim 14 wherein a plurality of coping strategies is retained by said scheduler and said monitor provides inputs to determine which of said coping strategies is appropriate.

16. The device of claim 15 wherein inputs to said monitor are compared to historical records to determine an appropriate coping strategy.

17. The device of claim 15 wherein response to a selected coping strategy is observed and said historical records updated.

18. The device of claim 1 wherein a plurality of coping strategies is retained by said scheduler and said monitor provides inputs to determine which of said coping strategies is appropriate.

19. The device of claim 18 wherein inputs to said monitor are compared to historical records to determine an appropriate coping strategy.

20. The device of claim 19 wherein response to a selected coping strategy is observed and said historical records updated.

21. The device of claim 3 wherein said threshold levels are modified by the scheduled activity.
